(19) United States
(12) Patent Application Publication
(10) Pub. No.: US 2006/0149132 A1
(43) Pub. Date: Jul. 6, 2006

(54) Device and method for in vivo illumination

(75) Inventor: Gavriel J. Iddan, Haifa (IL)

Correspondence Address: PEARL COHEN ZEDEK, LLP, 1500 BROADWAY 12TH FLOOR, NEW YORK, NY, 10036, US

(73) Assignee: GIVEN IMAGING LTD.

(21) Appl. No.: 11/024,753

(22) Filed: Dec. 30, 2004

(51) Int. Cl. A61B 1/06

(52) U.S. Cl. 600/160

(57) ABSTRACT

An in vivo imaging device including an illumination unit. The illumination unit may include, for,example, an illumination source such as an OLED.

### FIELD OF THE INVENTION

The present invention relates to a device useful for in-vivo imaging, more specifically to a device for providing illumination in-vivo.

### BACKGROUND OF THE INVENTION

Known devices may be helpful in providing in-vivo imaging. Autonomous in-vivo imaging devices, such as swallowable capsules or other devices may move through a body lumen, imaging as they move along. In vivo imaging may require in-vivo illumination, for example, using one or more light sources positioned inside an in-vivo imaging device.

### SUMMARY OF THE INVENTION

There is provided, in accordance with some embodiments of the present invention an in vivo imaging device having an illumination unit. According to one embodiment of the present invention the illumination unit may include, for example, a base or support for holding one or more light sources, for example, an organic light emitting diode (OLED) or other suitable illumination sources, that may enable in-vivo illumination.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIGS. 1A-1B show a schematic illustration of an in-vivo imaging device, according to one embodiment of the invention;

FIGS. 2A-2B show a schematic illustration of an illumination unit, according to one embodiment of the Invention;

FIG. 3 illustrates components of an OLED, according to one embodiment of the present invention;

FIG. 4 is a flowchart depicting a method for producing an illumination unit, according to embodiments of the invention;

FIG. 5 is a flowchart depicting a method for producing an in vivo device which includes an illumination unit, according to embodiments of the invention; and

FIG. 6 is a flowchart depicting a method for in vivo imaging, according to embodiments of the invention.

It should be noted that for simplicity and clarity of illustration, elements shown in the figures have not necessarily been drawn to scale. For example, the dimensions of some of the elements may be exaggerated relative to other elements for clarity. Furthermore, where considered appropriate, reference numerals may be repeated among the figures to Indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

Illumination sources used with embodiments of the present invention may include, for example, OLEDs or other suitable light sources that may enable in-vivo illumination with high luminous efficiency. An OLED is an electronic device that sandwiches carbon-based films between two charged electrodes, for example a metallic cathode and a transparent anode, usually being glass. Other embodiments may have other configurations and capabilities. OLED light sources may be lighter, thinner, more rugged, and more efficient than conventional lighting and/or light emitting diode (LED).

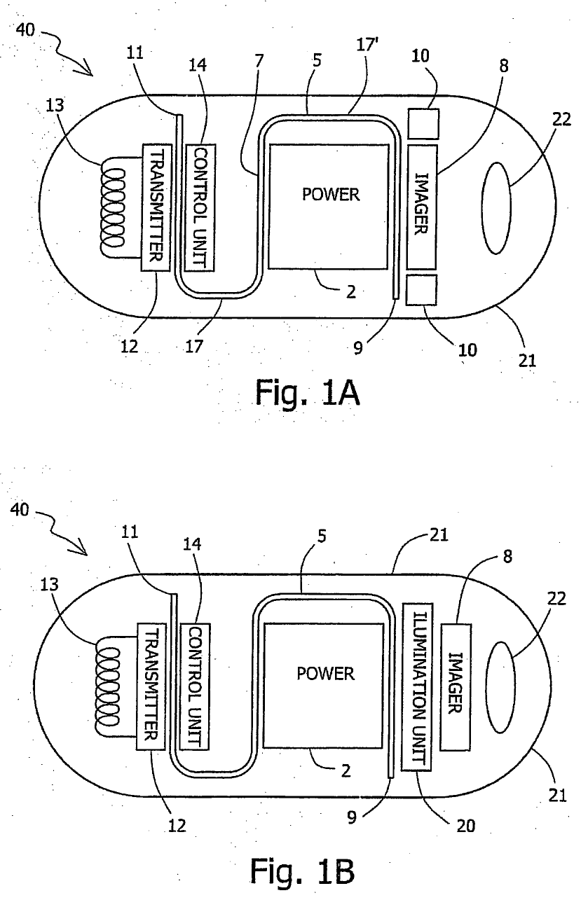

Reference is now made to FIG. 1A, which schematically illustrates an in vivo imaging device according to an embodiment of the invention. According to one embodiment the device 40 typically comprises an optical window 21 and an imaging system for obtaining images from inside a body lumen, such as the GI tract. The imaging system may include one or more illumination sources 10, such as a white LED and/or an OLED, an image sensor for example an imager 8, such as a CMOS imaging camera and an optical system 22 which focuses the images onto the imager 8. The illumination source 10 illuminates the inner portions of the body lumen through optical window 21. Device 40 may further include a control unit 14, a transmitter 12 and an antenna 13 for transmitting image signals from the imager 8, and a power source 2, such as a silver oxide battery, that provides power to the electrical elements of the device 10. A suitable imager 8 is, for example, a “camera on a chip” type CMOS imager specified by Given Imaging Ltd. of Yokneam, Israel and designed by Photobit Corporation of California, USA. Other suitable types of imagers may be used, for example, a CCD imager. The single chip camera can provide either black and white or color signals. A suitable transmitter may comprise a modulator which receives the image signal (either digital or analog) from the CMOS imaging camera, a Radio Frequency (RF) amplifier, an impedance matcher and an antenna. A processor, e.g., for processing the image data may be included in the device. The processor or processing circuitry may be integrated in the sensor or in the transmitter.

According to some embodiments the device 40 may be capsule shaped and can operate as an autonomous endoscope for imaging the GI tract. However, other devices, such as devices designed to be incorporated in an endoscope, catheter, stent, needle, etc., may also be used, according to embodiments of the invention. Furthermore, the device 40 need not include all the elements described above. For example, the device 40 need not include an internal light source or an internal power source; illumination and/or power may be provided from an external source, as known in the art.

According to one embodiment of the invention, the various components of the device 40 are disposed on a circuit board 5 including rigid and flexible portions; preferably the components are arranged In a stacked vertical fashion. For example, one rigid portion 11 of the circuit board 5 may hold a transmitter 12 and possibly an antenna 13; preferably the antenna is at one end of the device to avoid screening of the signal by metal or other components in the device. Another rigid portion 9 of the circuit board may include, for example, an illumination source 10, such as one or more LEDs, and/or OLEDs or other illumination source, and an imager 8 on one side; the other side of this rigid portion 9 may include, for example, a contact for battery or power source 2. According to one embodiment the battery contact is preferably a spring, such as described below. Another rigid portion 7 of the circuit board 5 may include, for example, another battery contact. Each rigid portion of the circuit board may be connected to another rigid portion of the circuit board by a flexible connector portion (e.g. 17 and 17′) of the circuit board. Preferably, each rigid portion of the circuit board may include two rigid sections; sandwiched between the rigid sections is a flexible connector portion of the circuit board for connecting the rigid boards. In alternate embodiments, other arrangements of components may be placed on a circuit board having rigid portions connected by flexible portions. In alternate embodiments, a circuit board having rigid portions and flexible portions may be used to arrange and hold components in other in vivo sensing devices, such as a swallowable capsule measuring pH, temperature or pressure, or in a swallowable imaging capsule having components other than those described above.

According to one embodiment, each flexible connector portion 17 and 17′ is equal to or less than 4/1000 inch (4 mils) in thickness. According to one embodiment, electrical connection is made from the outside portion of a rigid portion of a board (on which components are mounted) to the inside of the rigid portion and to the flexible portion contained within, by a small (equal to or less than 4 mils in diameter) hole leading from the outside portion to the flexible portion—e.g., a micro-via. The micro-via can be created using a laser. Companies providing such flexible connector and micro-via technology are Eltech, of Petach-Tikva, Israel, and Ilfa, of Germany. In alternate embodiments, other types of rigid sections and flexible sections may be used to create a circuit board.

The circuit board may be folded, for example, as shown in FIG. 1A. When folded, the battery contacts may contact a set of one or more batteries, e.g., power source 2, which may be sandwiched between two rigid circuit board portions. The circuit board may be folded in various manners. For example, FIG. 1A schematically shows a circuit board, according to an embodiment of the invention, arranged as an “S” with rigid portions 9, 17 and 11 and alternating flexible portions 17 and 17′.

Reference is now made to FIG. 1B, which illustrates components of an in-vivo sensing device, for example imaging device 40, according to some embodiments of the present invention. Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power from an external source or transmit information. Device 40 may communicate with an external receiving and display system to provide display of data, control, or other functions. For example, power may be provided by an internal battery or a wireless receiving system. Other embodiments may have other configurations and capabilities. For example, components may be distributed over multiple sites or units. Control information may be received from an external source.

Devices according to embodiments of the present invention, including imaging, receiving, processing, storage and/or display units suitable for use with embodiments of the present invention, may be similar to embodiments described in U.S. Pat. No. 5,604,531 to Iddan et al., and/or U.S. Patent Application, Pub. No. 2001/0035902 entitled A DEVICE AND SYSTEM FOR IN VIVO IMAGING, both of which are assigned to the common assignee of the present invention and which are hereby incorporated by reference. Of course, devices and systems as described herein may have other configurations and other sets of components.

In one embodiment, all of the components may be sealed within the device body (the body or shell may include more than one piece); for example, a control unit 14, an imager 8, an illumination unit 20, power source 2, and transmitting 12 and control 14 units, may all be sealed within the device body.

A single or plurality of light sources or a specific integrated light source, for example an OLED, may be used and positioned in the illumination unit 20 in accordance with specific imaging requirements, such as to avoid stray light etc.

According to one embodiment the device 40 may be capsule shaped and can operate as an autonomous endoscope for imaging the GI tract. However, other devices, such as devices designed to be incorporated in an endoscope, catheter, stent, needle, etc., may also be used, according to embodiments of the invention.

According to one embodiment of the invention, the various components of the device 40 are disposed on a circuit board 5, for example a flexible circuit board or a circuit board having rigid sections and flexible sections Such circuit boards may be similar to embodiments described in U.S. application Ser. No. 10/879,054 entitled IN VIVO DEVICE WITH FLEXIBLE CIRCUIT BOARD AND METHOD FOR ASSEMBLY THEREOF, and U.S. application Ser. No. 10/481,126 entitled IN VIVO SENSING DEVICE WITH A CIRCUIT BOARD HAVING RIGID SECTIONS AND FLEXIBLE SECTIONS, each incorporated by reference herein in their entirety. Preferably, according to one embodiment the components may be arranged in a stacked vertical fashion. For example, one portion 11 of the circuit board may hold a transmitter 12 and an antenna 13. Another portion 9 of the circuit board may include an illumination source, for example a rounded illumination unit 20.

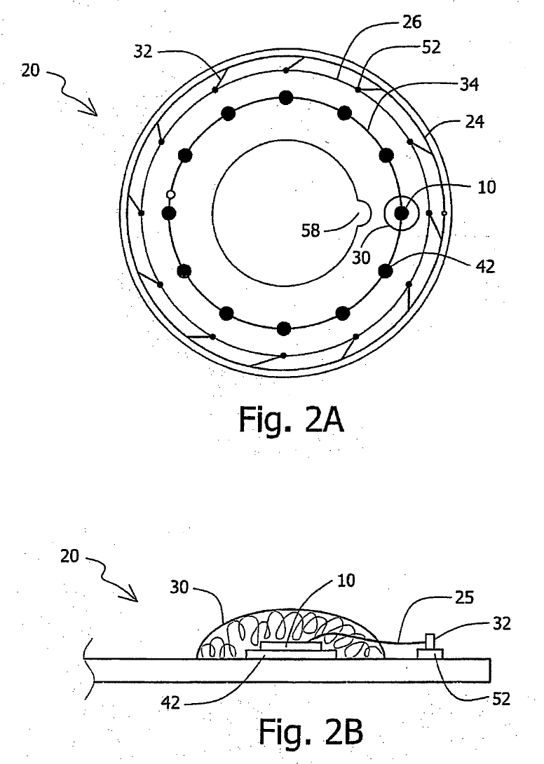

Reference is now made to FIG. 2A showing a schematic view from the top of the illumination unit 20 in accordance to one embodiment of the present Invention. According to one embodiment, the illuminating unit 20 may include one or more discrete light sources 10A, 10B, to 10L, for example OLEDs. Such OLED(s) may be similar to embodiments described in U.S. Pat. No. 6,579,629 entitled CATHODE LAYER IN ORGANIC LIGHT-EMITTING DIODE DEVICES and U.S. Pat. No. 4,720,432 entitled ELECTROLUMINESCENT DEVICE WITH ORGANIC LUMINESCENT MEDIUM, each incorporated by reference herein in their entirety. However, the light source(s) 10A, 10B, 10L of the illuminating unit 20 may also be any other suitable light source, known in the art, such as but not limited to monochromatic LED(s), incandescent lamp(s), flash lamp(s) or gas discharge lamp(s), or any other suitable light source(s).

According to some embodiments the illumination unit 20 may include a printed circuit board (PCB) made of, for example, silicone or plastic. Other suitable materials may be used. According to one embodiment the illumination unit 20 may be ring shaped for example with an internal circle e.g. a rounded hole 57 in its center. Typically, the illumination unit 20 has compatible measurements for a suitable incorporation into an in vivo device 40, for example an in vivo imaging device. The illumination unit 20 may be of a different shape other than a ring shape e.g. a rectangular or square shape, or of any other form compatible for fitting into an in vivo device.

According to one embodiment of the invention two printed traces 24 and 34, are printed on the illumination unit 20. Each of the printed traces 24 and 34 may be connected either to the positive terminal of the power source 2, or to the negative terminal of the power source 2 through printed trace 53 (shown in FIG. 2B). According to some embodiments of the invention another printed trace 26, which may be located, for example, between printed trace 24 and 34, may include a plurality of pads 52 for wire bonding, for example a plurality of resistors 32.

According to one embodiment of the present invention, conductive pads 42, for example metal pads for chip bonding may be placed or molded on printed trace 34, to provide connections for a plurality of discrete light sources 10A-10L, for example, to a number of OLEDs. Each light source 10A-10L may be associated with one or more additional components such as one or more resistor(s) 32, which may be connected to pad 26. Pad 26 may, for example, enable control over the amount of illumination generated by light sources 10A-10L. For example, a processor associated with device 40 may be able to use resistors 32 to generate different intensities of light in different parts of the GI tract, such as, 200 lux of light in the small intestine and 300 lux in the colon. Illumination may be controlled and customized for selected illumination functions. Resistor(s) 32 may be variable or permanent, for example a permanent resistor may enable normalized light output from a plurality of light sources.

According to one embodiment of the present invention, an optical resin 30 may be placed over each light source 10A-10L, for example over each OLED chip, providing different spectra of illumination (e.g., red, green or blue spectra, infra-red spectra or UV spectra). Furthermore, in certain embodiment, the various light sources 10A-10L may provide different spectra of illumination (e.g., red, green or blue spectra, infra-red spectra or UV spectra). In such embodiments, the illumination provided can be arranged in such a way that the illumination direction is different for each channel employing a different spectrum.

According to some embodiments, a depression 58, positioned in the internal circle of the illumination unit, serves as a direction marker during the illumination unit 20 installation within the in vivo device. In an alternate embodiment, depression 58 may be of other suitable shapes.

Reference is now made to FIG. 2B showing a schematic closer view from the side of a light source 10, for example an OLED, installed into the illumination unit 20, in accordance to one embodiment of the present invention. According to some embodiments the light source 10, may be placed over a conductive pad 42, for example a chip bonding pad, and may be connected through wire 25 to a pad 52, such as a pad for wire bond. According to some embodiments a resistor 32 may be placed on top of pad 52, for example, in order to control the light source 10 illumination intensity or other parameters such as amplitude.

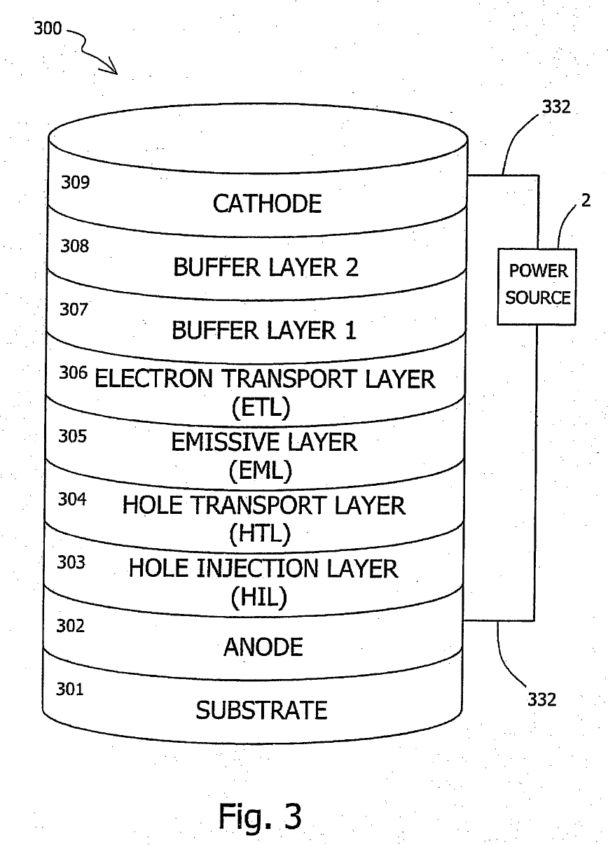

Reference is now made to FIG. 3, which illustrates components of an illumination source 300, for example an OLED, according to some embodiments of the present invention. According to some embodiments of the present invention, an OLED is a solid-state device made up of thin layers of organic films that emit bright light upon electrical stimulation. According to one embodiment of the present invention an OLED may include a substrate 301, an anode 302, a hole injection layer (HIL) 303, a hole transport layer (HTL) 304, an emissive layer (EML) 305, an electron transport layer (ETL) 306, two buffer layers 307 and 308, and a cathode 309. In operation, the anode and the cathode are connected to a voltage source, for example power source 2 via wires 332 and electrical current is passed through the organic layers, resulting in light emission or electroluminescence from the OLED device. Depending on the optical transparency of the anode and cathode, electroluminescence can be viewed from either the anode side or the cathode side. The intensity of the electroluminescence Is dependent on the magnitude of the electrical current that is passed through the OLED device, which in term is dependent on the luminescent and electrical characteristics of the organic layers as well as the charge injecting nature of the contacting electrodes. The composition and the function of the various layers constituting the OLED device may be similar to embodiments described in U.S. Pat. No. 6,679,629 entitled CATHODE LAYER IN ORGANIC LIGHT-EMITTING DIODE DEVICES which is hereby incorporated by reference. According to another embodiment of the present invention, an OLED may include a Metallic Contact layer, a polymer layer, an SiO₂ (Silicon Dioxide) layer, an Indium Tin Oxide layer and a Glass Substrate layer.

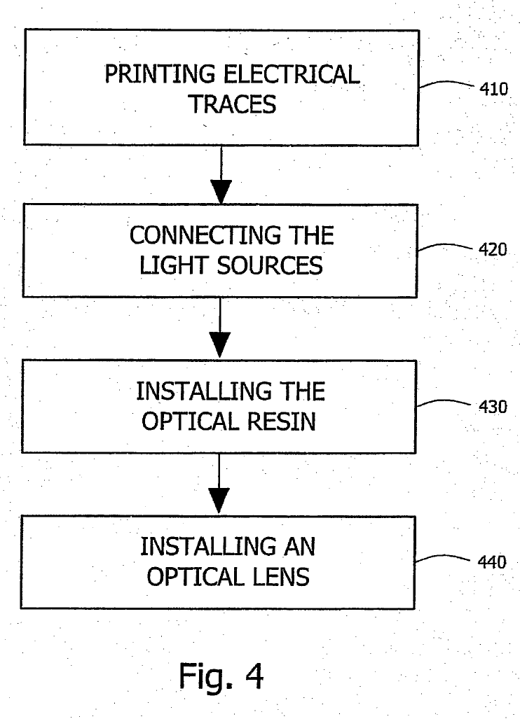

A method for producing an in vivo imaging device, which Includes a light source 10, for example an OLED, according to different embodiments of the invention is depicted in FIG. 4. According to some embodiments of the present invention, step 410 includes printing electrical traces on a substrate, such as a PCB. For example, a first electrical circuit, which may be wired to pads where the light sources 10, may be connected and a second electrical circuit which may be wired to pads where a plurality of resistors may be mounted on a PCB. Step 420 includes connecting the light sources, for example a plurality of OLEDS, to the resistors in order to be able to achieve different illumination intensities. Step 430 includes installing an optical resin above the light sources in order to create a vast spectrum of illumination inside the body. Step 440 includes installing an optical lens above the illumination unit in order to direct and focus the illumination.

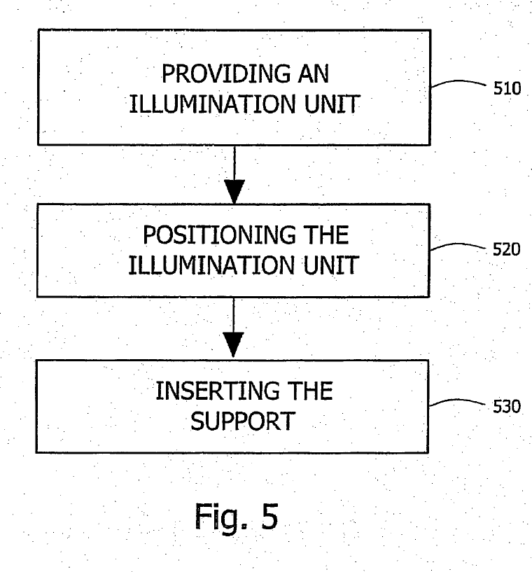

A method for providing in vivo illumination according to another embodiment is shown in FIG. 5. According to one embodiment the method may include providing an illumination unit (510) and positioning an illumination source, for example an OLED on the illumination unit (520), for example on a flexible PCB and inserting the illumination unit into a housing of an in vivo device (530).

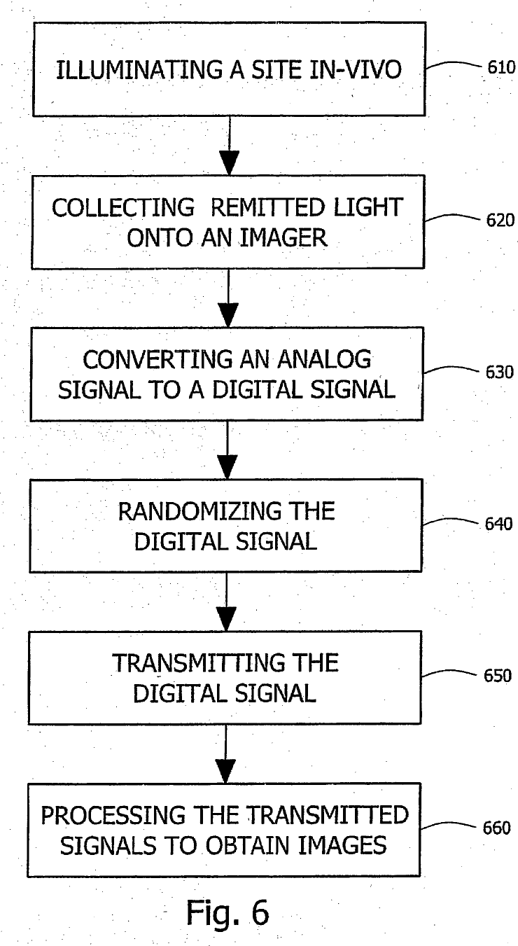

A method for providing in vivo illumination according to some embodiments of the present invention is shown in FIG. 6. The method for in vivo imaging includes the following steps: illuminating a site in vivo (610), for example by using an OLED; collecting remitted light onto an imager 8, thereby generating an analog signal (620); converting the analog signal to a digital signal (630); randomizing the digital signal (640); transmitting the digital signal to a receiving system (650) and processing the transmitted signals to obtain images of the in vivo site (660).

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is;

1. A device for in vivo imaging comprising an imager, an OLED, and an optical system.

2. The device according to claim 1 comprising a housing wherein the imager, OLED and optical system are contained within the housing.

3. The device according to claim 1, wherein a lens is mounted on the OLED.

4. The device according to claim 1, wherein said OLED include components selected from the group consisting of: a substrate; an anode; a hole injection layer, a hole transport layer; an emissive layer; an electron transport layer; a buffer layer and a cathode.

5. The device according to claim 1, wherein said OLED include components selected from the group consisting of: a Metallic Contact layer, a polymer layer, a SiO₂ layer, an Indium Tin Oxide layer and a Glass Substrate layer.

6. The device according to claim 1 comprising a power source.

7. The device according to claim 1 comprising a ring of OLEDs.

8. The device according to claim 1 wherein the OLED is positioned on a PCB.

9. The device according to claim 1 wherein the device is a capsule.

10. The device according to claim 1 comprising a transmitter.

11. A method for the manufacture of an in vivo imaging device comprising the steps of:
positioning an OLED on a support; and
folding said support into a device housing.

12. The method according to claim 11, providing an imager.

13. The method according to claim 11, providing a transmitting unit.

14. The method according to claim 11, providing a power source.

15. The method according to claim 11, providing a control unit.

16. The method according to claim 11, wherein said support is selected from the group consisting of: a flexible circuit board and a rigid-flex circuit board.

17. A method for the manufacture of an illumination unit for an in vivo imaging device, the method comprising the steps of:
printing electrical traces on a substrate,
disposing an OLED on said electrical traces; and
inserting the substrate in a housing of the in vivo imaging device.

18. The method according to claim 17, comprising installing an optical resin above said OLED.

19. The method according to claim 17, comprising installing a lens above said OLED.

20. The method according to claim 17, comprising installing a lens above a plurality of OLEDs.

21. The method according to claim 17, comprising installing a resistor on said electrical trace.
